(19) United States
(12) Patent Application Publication
(10) Pub. No.: US 2024/0415529 A1
(43) Pub. Date: Dec. 19, 2024

(54) ABLATION DEVICE AND METHOD OF USING THE SAME

(71) Applicant: Recor Medical, Inc., Palo Alto, CA (US)

(72) Inventor: Reinhard J. WARNKING, East Setauket, NY (US)

(21) Appl. No.: 18/814,283

(22) Filed: Aug. 23, 2024

Related U.S. Application Data
(63) Continuation of application No. 16/701,047, filed on Dec. 2, 2019, now Pat. No. 12,076,033.
Application No. 16/701,047 is a division of application No. 12/227,508, filed on Feb. 3, 2010, now Pat. No. 10,499,937.
(60) Provisional application No. 60/802,243, filed on May 19, 2006.

(51) Int. Cl. A61B 17/22; A61B 18/00; A61B 18/08; A61B 18/14

(52) U.S. Cl. CPC A61B 17/2202; A61B 18/082; A61B 18/1492; A61B 2017/22024; A61B 2017/22051; A61B 2018/00244; A61B 2018/00351; A61B 2018/00702; A61B 2018/00714

(57) ABSTRACT

Ablation device including a probe structure 10 having a proximal end 12 and a distal end 14. Probe structure 10includes a tubular first catheter 16, a tubular second catheter 18 surrounding the first catheter and a tubular guide catheter extending within the first catheter 16. The first catheter 16 carries a cylindrical ultrasonic transducer 20 adjacent its distal end. The transducer 20 is connected to a source of electrical excitation. The ultrasonic waves emitted by the transducer 20 are directed at the heart wall tissue. Once the tissue reaches the target temperature, the electrical excitation is turned on and off to maintain the tissue at the largest temperature. Alternatively, the transducer 20 is subjected to continuous excitation at one power level and upon the tissue reaching the target temperature, the power level of the continuous excitation is switched to a second lower power level.

### CROSS REFERENCE TO RELATED APPLICATIONS

The present application claims benefit of U.S. Provisional Application No. 60/802,243, filed May 19, 2006, the disclosure of which is hereby incorporated by reference herein.

### FIELD OF THE INVENTION

The present invention relates to medical procedures such as ablation and to apparatus and method for carrying out such procedures.

### BACKGROUND OF THE INVENTION

Ultrasonic heating such as high intensity focused ultrasound (HIFU) is utilized for certain therapeutic applications. As disclosed in commonly assigned International Application PCT/US98/1062, published as International Publication WO/98/52465 the disclosure of which is hereby incorporated by reference herein, HIFU heating typically is conducted using an ultrasonic emitter having an array of transducers. The transducers are actuated with a drive signal so as to emit ultrasonic waves. The relative phasing of the waves is controlled by the physical configuration of the array and the phasing of the drive signal. These factors are selected so that the ultrasonic waves tend to reinforce one another constructively at a focal location. Tissue at the focal location is heated to a greater extent than tissue at other locations. As described, for example in commonly assigned U.S. Pat. No. 6,461,314 and in commonly assigned U.S. Pat. No. 6,492,614, the disclosures of which are also incorporated by reference herein, HIFU may be applied by transducer arrays such as arrays of polymeric piezoelectric transducers. These arrays can be mounted on a probe such as a catheter which can be introduced into the body, for example, as in a cavernous internal organ or within the vascular system to perform cardiac ablation.

Contraction or “beating” of the heart is controlled by electrical impulses generated at nodes within the heart and transmitted along conductive pathways extending within the wall of the heart. Certain diseases of the heart known as cardiac arrhythmias involve abnormal generation or conduction of the electrical impulses. One such arrhythmia is atrial fibrillation or “AF.” Certain cardiac arrhythmias can be treated by deliberately damaging the tissue of the cardiac wall along a path crossing a route of abnormal conduction. This results in formation of a scar extending along the path where tissue damage occurred. The scar blocks conduction of the electrical impulses. Such a scar can be created by conventional surgery, but this entails all of the risks and expense associated with cardiac surgery. Alternatively, the scar may be made by application of energy such as heat, radio frequency energy or ultra sonic energy to the tissue that is to be scarred. Scarring the tissue by application of energy is referred to as cardiac ablation.

Commonly assigned U.S. Pat. No. 6,635,054, the disclosure of which is incorporated by reference herein in its entirety discloses thermal treatment methods and apparatus. The disclosed apparatus includes collapsible ultrasonic reflector. The reflector incorporates a gas-filled reflector balloon, a liquid-filled structural balloon and an ultrasonic transducer disposed within the structural balloon. Acoustic energy emitted by the transducer is reflected by a highly reflective interface between the balloons and focused into an annular focal region to ablate the cardiac tissue.

Commonly assigned U.S. Patent Application Publication No. US 2004/0176757, the disclosure of which is incorporated by reference herein in its entirety, discloses cardiac ablation devices. The disclosed devices are steerable and can be moved between a normal disposition, in which the ablation region lies parallel to the wall of the heart for ablating a loop like lesion, and a canted disposition, in which the ring-like focal region is tilted relative to the wall of the heart to ablate curved-linear lesions.

Conventional methods and apparatus, including the methods and apparatus mentioned above, utilize a continuous mode power profile to ablate cardiac tissue in the treatment of atrial fibrillation. However, with the conventional methods and apparatus, the collateral tissue immediately adjacent to the intended ablation target can heat up to a temperature that may result in unwanted necrosis of untargeted collateral tissue.

This unwanted necrosis of collateral tissue results from excess temperature elevation in the targeted tissue. Conventional systems deliver power in the continuous wave (CW) mode for the entire duration of the ablation cycle which sometimes results in temperature rises in the targeted tissue in excess of that needed to create necrosis. Heat from the target tissue is conducted to nearby collateral tissue and anatomical structures such as the phrenic nerve and esophagus. If the amount of heat energy is sufficiently high, than heat conducted from the targeted tissue to the collateral tissue results in elevated collateral tissue temperature sufficient to create unwanted necrosis.

Thus, there remains an unmet need for an optimized power delivery profile that quickly elevates the targeted tissue to temperatures resulting in necrosis, then maintains that temperature at a constant or near constant level for a period of time needed to achieve complete targeted tissue necrosis while, ensures that heat conducted to adjacent at the same time, collateral structures remain insufficient to cause unwanted or untargeted necrosis.

### SUMMARY OF THE INVENTION

One aspect of the present invention provides a method for applying energy within the body of a living subject. The method includes providing at least one energy emitter adapted to emit energy that impinges on a tissue within the body. The method further includes providing continuous power to the energy emitter for a first duration sufficient to reach a first temperature that is equal to or higher than the temperature necessary for necrosis of the tissue. Then, during a second state, the power to the energy emitter is switched on and off to substantially maintain the tissue at a second temperature.

A method according to a related aspect of the invention includes the steps of providing at least one energy emitter and directing the output of the energy emitter on a tissue within the The energy emitter is connected to power and the power body. turned on to emit energy at a first power level, for a first duration. The first duration is sufficient to reach a first temperature in the tissue, and the first temperature is equal to or higher than the temperature necessary for necrosis of the tissue. Next, the power is switched to emit energy at a lower power level. The lower power level is sufficient to substantially maintain the tissue at a second temperature.

Another aspect of the present invention provides an apparatus for applying energy within the body of a living subject. The apparatus includes an ultrasonic emitter and a housing for the ultrasonic emitter. The housing is adapted to place the ultrasonic emitter so that the output from the emitter will be directed to a tissue within the body. A power supply is connected to the ultrasonic emitter. The power supply is adapted to supply power to the ultrasonic emitter and thereby turn it on for a first duration sufficient for a tissue to reach a first temperature that is equal to or higher than the temperature necessary for necrosis of the tissue. Next, the power is cycle between on and off conditions to turn the ultrasonic emitter on and off to substantially maintain the tissue at a second temperature.

Apparatus according to further aspect of the invention includes an ultrasonic emitter and a housing for the ultrasonic emitter, the housing being adapted to place the ultrasonic emitter so that the output from the emitter will be directed to a tissue within the body. A power supply is connected to the ultrasonic emitter. The power supply is adapted to supply power to the ultrasonic emitter to emit ultrasonic energy at a first power level, for a first duration, the first duration being sufficient to reach a first temperature in the tissue, the first temperature being equal to or higher than the temperature necessary for necrosis of the tissue. Next, the ultrasonic emitter is powered to emit at a lower power level, the lower power level being sufficient to substantially maintain the tissue at a second temperature.

### BRIEF DESCRIPTION OF THE DRAWINGS

FIG. 1 is a diagrammatic view of apparatus in accordance with one embodiment of the invention in conjunction with a portion of a heart and pulmonary vein.

FIG. 2 is a diagrammatic sectional view taken along line 2--2 in FIG. 1.

FIG. 3 is a fragmentary diagrammatic view depicting certain geometrical relationships in the apparatus of FIG. 1.

FIG. 4 is an example of input power profile for the apparatus of FIG. 1.

FIG. 5 is a graph of temperature (at the phrenic nerve) versus time measured during animal experiments.

### DETAILED DESCRIPTION

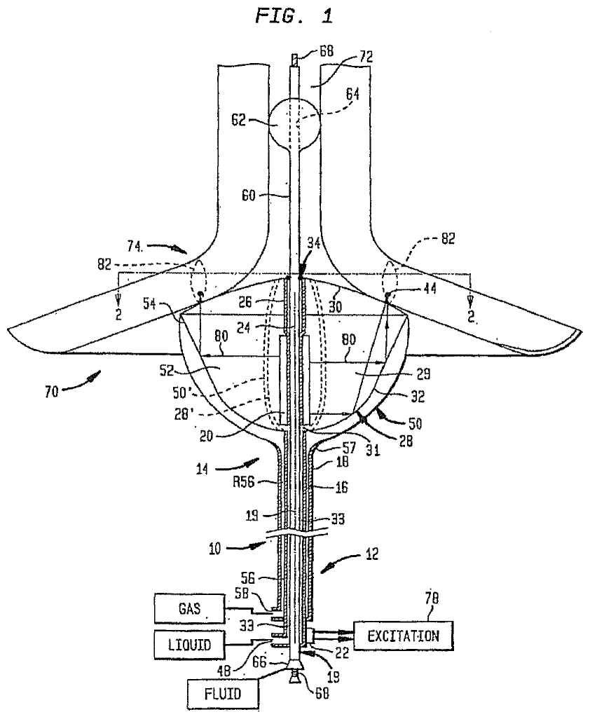

FIG. 1 shows one embodiment of ablation device of the invention. Many more embodiments of ablation device are disclosed in commonly assigned U.S. Pat. No. 6,635,054. Each of these embodiments can be used with the invention described herein. A portion of a probe structure 10 between proximal and distal ends 12 and 14 respectively is omitted in FIG. 1 for clarity of illustration. The probe structure includes a tubular first catheter 16 and a tubular second catheter 18 surrounding first catheter 16.

First catheter 16 and a cylindrical transducer 20 define a central axis 24 adjacent the distal end of the probe structure. First catheter 16 has a distal tip 26 projecting distally beyond transducer 20. A first balloon 28, also referred to herein as a “structural balloon,” is mounted to first catheter 16 at the distal end thereof. First balloon 28 includes an active wall 32 formed from film which is flexible but which can form substantially noncompliant balloon structure when inflated. A forward wall 30 may be generally conical or dome-shaped and may project forwardly from its juncture with active wall 32. For example, forward wall 30 may be conical, with an included angle of about 120 degrees. Forward wall 30 joins the wall of first catheter 16 at distal tip 26 thereof, whereas active wall 32 joins the wall of catheter 16 proximally of transducer 20. Thus, transducer 20 is disposed inside of first balloon 28.

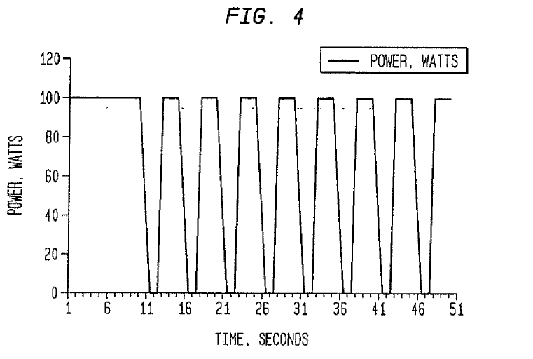

The shape of active wall region 32 depicted in FIG. 1 may be that of a surface of revolution about central axis 24 formed by rotation of a generatrix or curve 38 (FIG. 3) which is a portion of a parabola 40 having its principal axis 42 transverse to and desirably perpendicular to central axis 24. Focus 44 of the parabolic generatrix is slightly forward or distal of forward wall 30 when the balloon is in the inflated condition.

A second balloon 50, also referred to herein as the “reflector balloon,” is carried on the distal end of second catheter 18. When both first and second balloons 28 and 50, respectively, are in a deflated position, second balloon 50 is collapsed inwardly, toward central axis 24 so that second balloon 50 in deflated condition 50′ closely overlies deflated first balloon 28′.

In use, the probe structure, with first balloon 28 and second balloon 50 deflated, is threaded through the subject's circulatory system. Thereafter, upon inflation of first balloon 28 and second balloon 50, forward wall 30 of first balloon 28 bears on the interior surface of the heart wall at an ostium or opening 74 at which pulmonary vein 72 communicates with heart chamber 70.

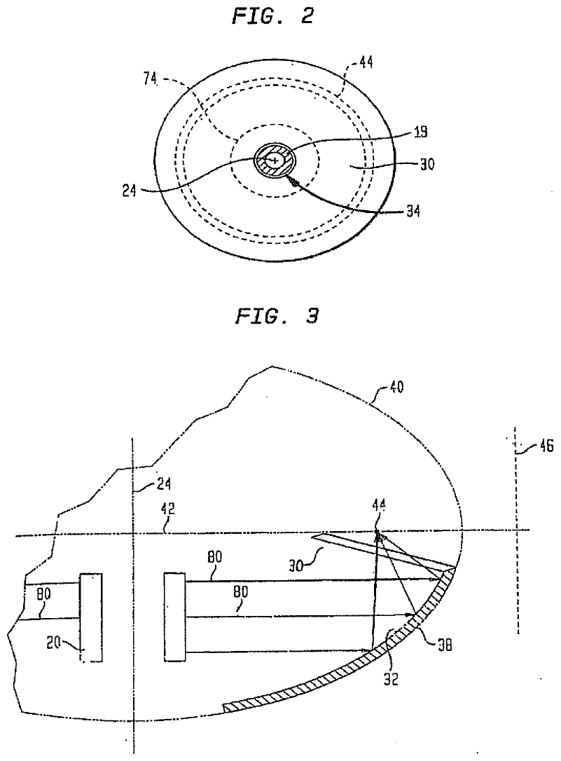

Transducer 20 is connected to a source 78 of electrical excitation signals through connector 22. Source 78 is adapted to provide continuous and intermittent electrical excitation. Thus, Source 78 can provide continuous excitation for a predetermined period of time and then turn the electrical excitation on and off for a predetermined period of time. For example, after providing continuous excitation for between 5 and 30 seconds, source 78 may turn the electrical excitation off for a one second and then turn it on for one second and repeat the on-off cycle for a predetermined period of time. The electrical excitation actuates transducer 20 to produce ultrasonic waves. The ultrasonic waves propagate substantially radially outwardly as indicated by arrows 80 in FIGS. 1 and 3. Stated another way, cylindrical transducer 20 produces substantially cylindrical wave fronts which propagate generally radially outwardly. These waves are reflected by the interface at active region 32. Because the interface has a parabolic shape, the waves striking any region of the interface will be reflected substantially to focus 44 defined by the surface of revolution, i.e., into a substantially annular or ring-like focal region at focus 44. As best seen in FIG. 2, this ring-like focus surrounds central axis 24 and surrounds ostium 74 of the pulmonary vein. This focal region is slightly forward of forward wall 30 and hence within the heart tissue, near the surface of the heart wall. For example, the focal region may be disposed at a depth equal to about one-half of the thickness of the heart wall as, for example, about 2-4 mm from the surface of the wall.

The heart wall tissue located at focus 44 is heated rapidly. The initial CW power delivery is performed with high power output to quickly create the initial lesion which creates an absorptive barrier for ultrasound and therewith protects distal collateral structures. It is believed that the lesion will mostly grow towards the source. The temperature of the tissue depends upon several factors including the output power of transducer 20 and the time for which the tissue is exposed to the output of transducer 20. Upon the target tissue being exposed to the ultrasonic output of transducer 20 for a predetermined time, the target tissue reaches the target temperature, i.e., the temperature that would result in necrosis. The target temperature may be in the range 55-80 degrees centigrade, preferably in the range 55-60 degrees centigrade. The continuous excitation is maintained for a first duration sufficient for the target tissue to reach the target temperature. At the end of the first duration, the electrical excitation is turned on and off to maintain the target tissue at the target temperature. The rapid heating of the target tissue to the target temperature effectively ablates or kills the tissue at the focal region so that a wall of non-conductive scar tissue forms in the focal region and in neighboring tissue. However, by turning the electrical excitation on and off and thereby maintaining the target tissue the target temperature, the amount of at neighboring that is killed is minimized. This is in contrast to keeping the electrical excitation on continuously for the entire duration of time necessary to ablate the target tissue. If the electrical excitation is kept on for the entire duration of time necessary to ablate the target tissue, the temperature of the target tissue keeps rising for the entire duration and exceeds the temperature necessary for tissue necrosis. This results in necrosis of greater amount of neighboring tissue as compared to when the electrical excitation is turned on and off during the ablation cycle. For a particular ablation apparatus using particular transducer, the time it takes for the target tissue to reach the target temperature may be determined via theoretical models or experimentally or by a combination of these techniques. For a given ablation apparatus, experiments may be carried out wherein the cardiac tissue is ablated and temperature of the tissue at different time measured by known techniques such as use of thermocouples or imaging. Based upon these experiments, a recommendation for duration of operation of the ablation apparatus in the continuous mode and duration of operation in the on-off mode would be provided to the physicians. The process will have to be repeated for an ablation apparatus of a different design.

FIG. 4 shows the input power profile for electrical excitation of transducer 20. At the beginning of the ablation cycle, transducer 20 is supplied with 100 watt electrical excitation signal. The excitation of transducer 20 is continuous for approximately 11 seconds. However, the continuous excitation mode may range from 5 seconds to 30 seconds or for the duration necessary for the target tissue to reach the target temperature. Once the target tissue has reached the target temperature the input power is cycled off and on as shown in FIG. 4. The power is off for roughly 25 percent of the time and the power is on for roughly 75 percent of the time. To put it another way, once the target tissue has reached the target temperature, the power is off for roughly 1 second and then it is on for roughly 3 seconds with the on-off cycle continuing for the duration of the ablation cycle. The power is turned off at the end of the ablation cycle and the temperature of the target tissue drops rapidly thereafter. It should be noted that the power on-off cycle can be varied, for example, similar results may be obtained with the power being off for one second and on for one second following the continuous excitation mode. Alternatively, the entire power input profile may consist of continuous excitation at one power level and upon reaching of the target temperature switching to continuous excitation at a second lower power level.

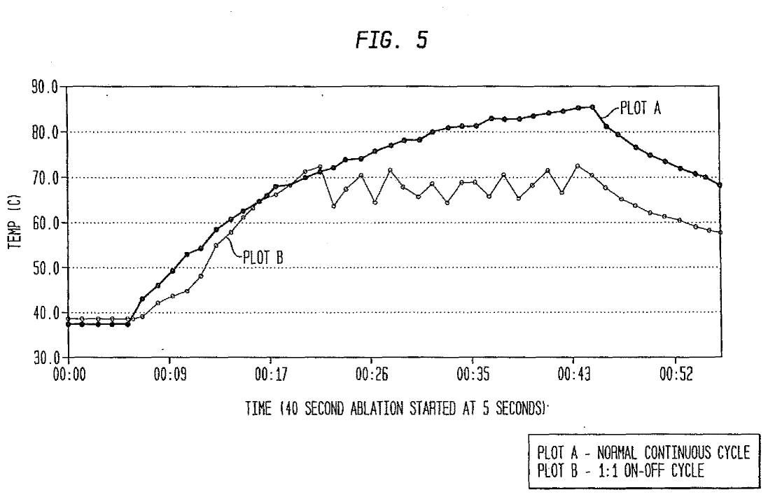

FIG. 5 shows plots of temperature measured at a fixed distance from the target tissue during animal experiments. A 20 mm HIFU Ablation Catheter made by Prorhythm, inc., was used. The Ablation Catheter was supplied with 100 watt electrical excitation signal, and the HIFU output was 32 watts acoustic. A 20 mm ablation device kills tissue in the shape of a ring of 20 mm diameter. Several embodiments of ablation devices that can cause necrosis of tissue in shape of a ring are disclosed in U.S. Pat. No. 6,635,054 and U.S. Patent Application Publication No. US 2004/0176757. As seen in FIG. 5, plot A, the temperature at the fixed distance from the target tissue keeps rising for the entire duration of the ablation cycle when the electrical excitation is kept on for the entire duration of the ablation cycle. On the other hand, as seen in FIG. 5, plot B, when the electrical excitation is turned on and off during a portion of the ablation cycle, the temperature at the fixed distance from the target tissue rises during the continuous excitation mode and then remains substantially constant at that level during the on-off mode. This results in reduction or elimination of collateral tissue damage.

Some of the ultrasonic energy is absorbed between the surface of the wall and the focal region, and at locations deeper within the wall than the focal region. To provide a complete conduction block, tissue should be ablated through the entire thickness of the wall, so as to form a transmural lesion. With a transducer capable of emitting about 15 Watts of acoustic energy, an ablated region extending entirely through the heart wall can be formed within a few minutes of actuation. Higher power levels as, for example, above 30 Watts of acoustic energy and desirably about 45 Watts are preferred because such power levels will provide shorter lesion formation time (under one minute). Because the sonic energy is directed simultaneously into the entire loop-like path surrounding the pulmonary vein, the PV isolation can be performed ideally without repositioning the probe. However, several applications may be required due to non circular, irregular anatomy.

The positioning of the ablation device within the heart desirably includes selectively controlling the disposition of the forward-to-rearward axis 24 of the device relative to the patient's heart. That is, the position of the forward-to-rearward axis desirably can be controlled by the physician to at least some degree. To that end, the assembly can be provided with one or more devices for selectively bending the ablation device. Various embodiments of the ablation device that lend themselves to allow disposition of the ablation device to be selectively controlled are disclosed in commonly assigned Patent Application No. US 2004/0176757. Each of these embodiments may be used in conjunction with the input power profile disclosed herein. Although the invention has been described with the aid of an ablation device using HIFU, any form of output power for ablating the tissue may be used in the on-off mode as described herein to realize the benefit of the invention. Non limiting examples of the other forms of output power are RF and heat.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### INDUSTRIAL APPLICABILITY

This application relates to the medical device industry.

1-20 (canceled)

21. An apparatus for applying energy within the body of a living subject, comprising:
an ultrasonic emitter configured for placement inside the body;
a housing for the ultrasonic emitter, the housing configured to place the ultrasonic emitter so that an output from the ultrasonic emitter is directed to a tissue within the body; and
a power supply connected to the ultrasonic emitter;
wherein the power supply comprises a source adapted to supply power to the ultrasonic emitter according to an input power profile, wherein the ultrasonic emitter emits ultrasonic energy into the tissue when power is supplied thereto, and wherein the profile comprises:
a first predetermined duration, during which a continuous excitation to the ultrasonic emitter is provided, which causes the ultrasonic emitter to emit ultrasonic energy into the tissue for the tissue to reach a first temperature that is equal to or higher than the temperature necessary for necrosis of the tissue, and
a second predetermined duration, during which an intermittent excitation to the ultrasonic emitter is provided to cycle between on and off conditions to turn the ultrasonic emitter on and off, so as to substantially maintain the tissue at a second temperature.

22. The apparatus of claim 21, wherein the first temperature is around 55-60 degrees Celsius.

23. The apparatus of claim 22, wherein the second temperature is equal to the first temperature.

24. The apparatus of claim 22, wherein the second temperature is around 55-60 degrees Celsius.

25. The apparatus of claim 21, wherein the first duration is between 5 seconds and 30 seconds.

26. The apparatus of claim 21, wherein the power supply is adapted to cycle between on and off conditions and the power to the ultrasonic emitter is off for 25 percent of the second predetermined duration.

27. The apparatus of claim 21, wherein the power supply is adapted to cycle between on and off conditions and the power to the ultrasonic emitter is on for 75 percent of the second predetermined duration.

28. The apparatus of claim 21, wherein the power supply is adapted to cycle between on and off conditions and the power cycles between being off for 1 second and on for 3 seconds during the second predetermined duration.

29. The apparatus of claim 21, wherein the second temperature is different from the first temperature.

30. The apparatus of claim 21, wherein the housing is a balloon.

31. The apparatus of claim 21, wherein the ultrasonic emitter is cylindrical.

32. An apparatus for applying energy within the body of a living subject, comprising:
an energy emitter configured for placement inside the body; and
a power supply connected to the energy emitter;
wherein the power supply comprises a source of electrical signals adapted to supply input power to the energy emitter according to an input power profile, wherein the energy emitter emits energy into the tissue when power is supplied thereto, and wherein the profile comprises:
a first predetermined duration, during which input power is applied from the power supply to the energy emitter, which in turn emits output power into the tissue, causing the tissue to reach a first temperature that is equal to or higher than the temperature necessary for necrosis of the tissue, and
a second predetermined duration, during which input power is applied from the power supply to the energy emitter, which in turn emits output power into the tissue to maintain the tissue substantially at a second temperature.

33. The apparatus of claim 32, wherein the energy emitter is an ultrasound transducer, and wherein the ultrasound transducer emits ultrasound energy into the tissue when power is supplied thereto.

34. The apparatus of claim 32, wherein the energy emitter emits RF energy, and wherein the energy emitter emits RF energy into the tissue when power is supplied thereto.

35. The apparatus of claim 32, wherein during the first predetermined duration, the output power is substantially continuous.

36. The apparatus of claim 32, wherein during the second predetermined duration, the output power is intermittent.

37. The apparatus of claim 32, wherein during at least one of the first predetermined duration and the second predetermined duration, the output power is noncontinuous.

38. The apparatus of claim 32, wherein the output power is ultrasound.

39. The apparatus of claim 32, wherein the output power is RF.

40. The apparatus of claim 32, wherein the second temperature is different from the first temperature.
